(19) United States
(12) Patent Application Publication
(10) Pub. No.: US 2021/0265146 A1
(43) Pub. Date: Aug. 26, 2021

(54) IMAGING MASS SPECTROMETRY METHOD

(71) Applicant: SEKISUI MEDICAL CO., LTD., Tokyo (JP)

(72) Inventors: Masashi HIRAMOTO, Ushiku-shi, Ibaraki-ken (JP); Toshimasa ITO, Tokyo (JP)

(73) Assignee: SEKISUI MEDICAL CO., LTD., Tokyo (JP)

(21) Appl. No.: 17/252,001

(22) PCT Filed: Sep. 28, 2018
(86) PCT No.: PCT/JP2018/036222

§ 371 Date: Dec. 14, 2020

(30) Foreign Application Priority Data
Jun. 15, 2018 (JP) 2018-114079

(51) Int. Cl. H01J 49/00; G01N 33/68; H01J 49/16

(52) U.S. Cl. CPC H01J 49/0004; H01J 49/164; H01J 49/0031; G01N 33/6806

(57) ABSTRACT

A problem to be solved by the present invention to perform an imaging mass spectrometry method capable of not only providing localization information of a biological substance in a single tissue section but also enabling quantitative comparison of an amount of a biological substance between multiple tissue sections.

By using a first derivatization reagent, a second derivatization reagent that is the isotopically-labeled first derivatization reagent, and a derivatized internal standard substance of a biological substance in the imaging mass spectrometry method, the amount of the biological substance can quantitatively be compared between multiple tissue sections.

### TECHNICAL FIELD

The present invention relates to an imaging mass spectrometry method capable of quantifying a biological substance in a biological sample. The present invention also relates to a kit used for quantitatively comparing a biological substance in a biological sample in an imaging mass spectrometry method.

### BACKGROUND ART

Biological substances existing in the body of an organism include an endogenous substance derived from metabolism of the organism, a chemical substance derived from a microorganism symbiotic with the organism, and a drug or chemical substance ingested by or administered to the organism and metabolites thereof. Mass spectrometry is often used to analyze amounts of these biological substances.

Mass spectrometry is a technique for measuring a mass-to-charge ratio of molecules or atoms to obtain information such as molecular weight or atomic weight. Among the mass spectrometry methods, imaging mass spectrometry is a technique of performing localization analysis of a target substance based on a mass spectrum obtained by directly performing mass spectrometry on a surface of a tissue section. The imaging mass spectrometry is widely used for pharmacokinetic analysis, biomarker search, etc. However, while the imaging mass spectrometry can provide distribution information of a target substance in a tissue, the imaging mass spectrometry has a drawback that it is difficult to provide quantitative information of a target substance in a tissue. Specifically, due to the presence of an ionization inhibitor, an amount of ionization during laser irradiation varies between sample sections or tissue sites, which makes it difficult to quantitatively compare an amount of a biological substance between tissue sections using imaging mass spectrometry.

Various techniques have been proposed to overcome the drawback. For example, in a known method, a frozen section is prepared after a known amount of an object to be measured is added to a homogenate of a tissue that is a specimen so as to create a calibration curve (Non-Patent Document 1). However, this technique requires preparation of a considerable number of frozen sections at various concentrations for creation of the calibration curve, which makes the operation complicated. Additionally, it is expected that this technique has difficulty in quantifying a small variation such as two-fold variation and cannot correct a difference in suppression of ionization in mass spectrometry between tissue sites.

Patent Document 1 discloses that a substance in hair is analyzed by performing imaging mass spectrometry using an internal standard. However, this technique cannot be used for substances hardly ionized by laser irradiation, and the substances to be analyzed are significantly limited.

It is also reported that biological substances are quantitatively compared between tissue sections using stable isotope-labeled biological substances containing a stable isotope in the chemical structure of the biological substances (Non-Patent Document 2). However, in this case, quantitative comparison between tissue sections may be enabled only for a small portion of biological substances for which the stable isotope-labeled substances are commercially available. Moreover, when a mass difference between the stable isotope-labeled substances is small, the biological substance itself may be contained as an impurity. Therefore, in the case of a biological substance for which a stable isotope-labeled substance is not commercially available, it is difficult to quantitatively compare the biological substance between tissue sections.

On the other hand, derivatization reagents are reagents for derivatizing a substance hardly ionized in ordinary measurement in mass spectrometry so as to facilitate ionization. The derivatization reagents are used for the purpose of improving detection sensitivity in mass spectrometry.

### CITATION LIST

### Patent Literature

Patent Document 1: Japanese Laid-Open Patent Publication No. 2014-52322

### Non Patent Literature

Non-Patent Document 1: Takai, N et al., Mass Spectrom. Vol. 3 (2014), A0025, DOI: 10.5702/massspectrometry. A0025
Non-Patent Document 2: Bergman H M et al., Analyst, 2016, 141, 3686-3695.

### SUMMARY OF INVENTION

### Technical Problem

As described above, although localization analysis of a biological substance can be performed in a single tissue section, imaging mass spectrometry has a drawback that the amount of the biological substance cannot quantitatively be compared between multiple tissue sections. Therefore, a demand exists for an imaging mass spectrometry method capable of not only providing localization information of biological substances in a single tissue section but also enabling quantitative comparison of a wide range of biological substances between multiple tissue sections with simple operation.

### Solution to Problem

As a result of intensive studies for solving the problem, the present inventor founds that by using a first derivatization reagent, a second derivatization reagent that is the isotopically-labeled first derivatization reagent, and an internal standard substance of a biological substance in an imaging mass spectrometry method, the imaging mass spectrometry method capable of quantitatively comparing an amount of a biological substance between multiple tissue sections can be performed with simple operation, thereby completing the present invention.

Specifically, the present invention is as follows.

<1> An imaging mass spectrometry method of a biological substance in multiple sample sections, the method comprising the steps of:

(A) bringing a first derivatization reagent into contact with the biological substance in a first sample section;

(B) applying an internal standard substance of the biological substance to the first sample section, the internal standard substance being derivatized with a second derivatization reagent;

(C) bringing the biological substance and the internal standard substance on the first sample section into contact with a matrix to obtain a mass spectrum;

(D) bringing the first derivatization reagent or the second derivatization reagent into contact with the biological substance in a second sample section;

(E) applying the internal standard substance of the biological substance to the second sample section, the internal standard substance being derivatized with the first derivatization reagent or the second derivatization reagent not used in the step (D);

(F) bringing the biological substance and the internal standard substance on the second sample section into contact with the matrix to obtain the mass spectrum; and

(G) quantitatively comparing a spectral intensity ratio of the derivatized biological substance and the derivatized internal standard substance between the first sample section and the second sample section,

wherein the second derivatization reagent is the first derivatization reagent labeled with an isotope.

<2> The imaging mass spectrometry method according to <1>, wherein the first derivatization reagent is used in the step (D), and wherein the second derivatization reagent is used in the step (E).

<3> The imaging mass spectrometry method according to <1> or <2>, wherein the first derivatization reagent and the second derivatization reagent are an amino group modification reagent respectively, and wherein the biological substance in the first and second sample sections is a substance having an amino group.

<4> The imaging mass spectrometry method according to <3>, wherein the substance having the amino group is an α-amino acid or a derivative thereof.

<5> The imaging mass spectrometry method according to <3> or <4>, wherein the first derivatization reagent and the second derivatization reagent are mTRAQ (registered trademark) reagents having different molecular weights respectively.

<6> The imaging mass spectrometry method according to any one of <1> to <5>, wherein the sample sections are derived from human or animal biological tissues.

<7> The imaging mass spectrometry method according to <6>, further comprising:

(H) a step of treating a biological tissue extract with the second derivatization reagent to obtain a first extract sample containing a derivatized internal standard substance, and

(I) a step of treating the biological tissue extract with the derivatization reagent used in the step (E) to obtain a second extract sample containing the derivatized internal standard substance,

wherein the first extract sample is applied to the first sample section in the step (B), and wherein the second extract sample is applied to the second sample section in the step (E).

<8> The imaging mass spectrometry method according to any one of <1> to <7>, wherein two or more internal standard substances are applied to the first sample section in the step (B), and wherein two or more internal standard substances are applied to the second sample section in the step (E).

<9> A quantification kit for a biological substance in a living body, comprising: a first derivatization reagent for labeling and derivatization of the biological substance;

and
an internal standard substance of the biological substance, which is derivatized with a second derivatization reagent,

wherein an imaging mass spectrometry is used for quantification of the biological substance, and wherein the second derivatization reagent is the first derivatization reagent labeled with an isotope.

<10> The quantification kit according to <9>, wherein the first derivatization reagent and the second derivatization reagent are an amino group modification reagent respectively, and wherein the biological substance in the living body is a substance having the amino group.

<11> The quantification kit according to <10>, wherein the substance having the amino group is an amino acid or a derivative thereof.

<12> The quantification kit according to <10> or <11>, wherein the first derivatization reagent and the second derivatization reagent are mTRAQ (registered trademark) reagents having different molecular weights respectively.

### Advantageous Effects of Invention

The present invention can provide the imaging mass spectrometry capable of not only providing localization information of a biological substance in a single tissue section but also enabling quantitative comparison of an amount of a biological substance between multiple tissue sections. According to the present invention, the amount of the biological substance can quantitatively be compared between a tissue section of a pathological model mouse and a tissue section of a normal model mouse. According to the present invention, pharmacokinetics of a drug administered to animals can quantitatively be compared between a pathological model mouse and a normal model mouse.

### BRIEF DESCRIPTION OF DRAWINGS

FIG. 1 is a schematic diagram showing a procedure of acquiring distribution information of a biological substance according to conventional imaging mass spectrometry.

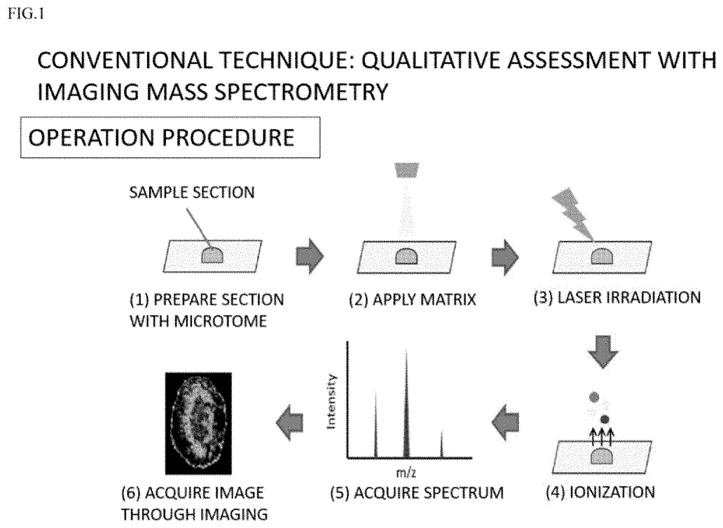

FIG. 2 is a schematic diagram showing a procedure of acquiring localization and quantitative information of a biological substance (endogenous amine) according to an embodiment of an imaging mass spectrometry of the present invention.

FIG. 3 is a schematic diagram showing a procedure of acquiring localization and quantitative information of a biological substance (endogenous GABA) according to an embodiment of the imaging mass spectrometry of the present invention.

FIG. 4 is a schematic diagram showing a derivatization reaction of a biological substance on a tissue section according to an embodiment of the imaging mass spectrometry of the present invention.

FIG. 5 is a schematic diagram showing a quantitative comparison between multiple tissue sections according to an embodiment of the imaging mass spectrometry of the present invention.

FIG. 6 is a photograph showing results of imaging mass spectrometry of Example 1 using a control rat and a stroke model rat.

FIG. 7 is a schematic diagram showing a procedure of a comprehensive analysis of amounts of multiple biological substances in a living body according to an embodiment of the imaging mass spectrometry of the present invention.

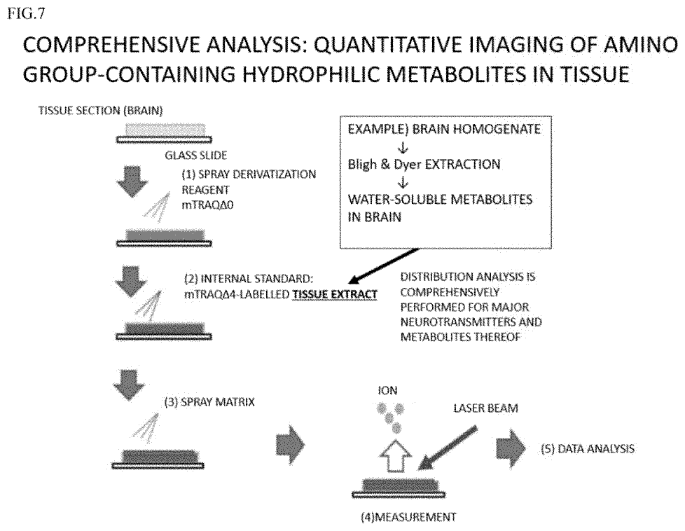

FIG. 8 is a photograph showing a result of a comprehensive analysis of amounts of multiple biological substances in a living body using a control rat.

### DESCRIPTION OF EMBODIMENTS

### [1] Imaging Mass Spectrometry Method

### (First Derivatization Reagent and Second Derivatization Reagent)

An imaging mass spectrometry method of the present invention uses a first derivatization reagent and a second derivatization reagent. One of the first derivatization reagent and the second derivatization reagent is used for the purpose of derivatization of a substance (biological substance) existing in a sample, and the other is used for the purpose of derivatization of an internal standard substance of the biological substance.

In this description, a derivatization reagent is a reagent for derivatizing a substance that is hardly ionized in ordinary measurement in mass spectrometry so as to facilitate ionization. In the imaging mass spectrometry method of the present invention, one of the first derivatization reagent and the second derivatization reagent is brought into contact with a biological substance to derivatize a specific functional group of the biological substance, and the biological substance is derivatized. Examples of the specific functional group reactive with the derivatization reagent include an amino group, a hydroxyl group, a thiol group, a carboxy group, a formyl group, a carbonyl group, an amide, an ester, etc. The specific functional group reactive with the derivatization reagent is preferably an amino group or a carboxy group, more preferably an amino group.

In this description, the amino group means monovalent functional groups obtained by removing hydrogen from ammonia, primary amine, and secondary amine, which are represented by —NH₂, —NHR, and —NRR″, respectively.

In the imaging mass spectrometry method of the present invention, the first derivatization reagent and the second derivatization reagent are selected in consideration of the chemical structure and properties of the target biological substance and are not particularly limited as long as the derivatization reagents can derivatize the target biological substance and the internal standard substance and generate a mass difference between the biological substance and the internal standard substance. The first derivatization reagent and the second derivatization reagent are preferably amino group- or carboxy group-modification reagents, more preferably amino group-modification reagents, and most preferably mTRAQ (registered trademark) (mTRAQΔ0, mTRAQΔ4, and mTRAQΔ8) reagents having respective different molecular weights. The first derivatization reagent and the second derivatization reagent are preferably combined such that the derivatized biological substance and the derivatized internal standard substance have a mass difference of 4 or more.

The structural formula of mTRAQ (registered trademark) reagent is as follows.

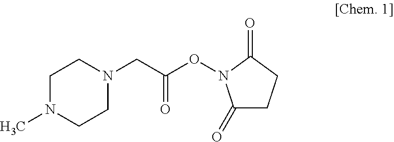 [SMILES: CN1CCN(CC(=O)ON2C(=O)CCC2=O)CC1]

Three types of the reagents mTRAQΔ0, mTRAQΔ4, and mTRAQΔ8 have different molecular weights depending on the type and number of labeling isotopes and labeled sites. For example, when dopamine is derivatized with the mTRAQ (registered trademark) reagents, the molecular weights of mTRAQΔ0 dopamine, mTRAQΔ4 dopamine, and mTRAQΔ8 dopamine are 293, 297, and 301, respectively, as integer values. These derivatized dopamines generate different spectra in mass spectrometry based on differences in molecular weight.

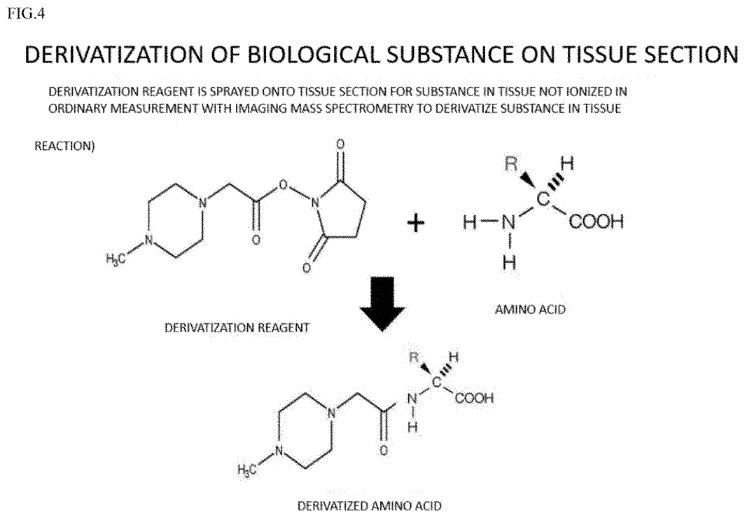

The reaction in the case of derivatizing an amino acid with the mTRAQ (registered trademark) reagents is shown in FIG. 4.

By derivatizing the biological substance and the internal standard substance with respective derivatization reagents having different masses and using the derivatized internal standard substance, the distribution of the biological substance in multiple tissue sections can quantitatively be compared. The biological substance and the internal standard substance are respectively reacted with the first derivatization reagent and the second derivatization reagent and are changed into two types of substances having the same ease of ionization and different masses. Therefore, in the imaging mass spectrometry method of the present invention, a spectral intensity ratio between the derivatized biological substance and the derivatized internal standard substance can be calculated on the basis of the mass difference between the derivatization reagents. Based on this intensity ratio, a difference in amount of ionization due to the presence of an ionization inhibitor etc. can be corrected. The imaging mass spectrometry method of the present invention not only provides the distribution information in a single tissue section but also enables quantitative comparison of the amount of biological substance between multiple tissue sections based on this intensity ratio.

Regarding the derivatization reagents, already isotopically-labeled commercially available derivatization reagents may be purchased, or commercially available derivatization reagents may be labeled with an isotope.

In the imaging mass spectrometry method of the present invention, the first derivatization reagent and the second derivatization reagent are used in one measurement. In this description, “one measurement” means a series of operations and measurements for analyzing a specific biological substance by using the imaging mass spectrometry method. For example, in the case that MALDI (Matrix Assisted Laser Desorption/Ionization) is selected as the ionization method, the one measurement means a series of operations and measurements for preparation of a sample section, application of matrix, ionization by laser irradiation, and acquisition of mass spectrum.

### (Sample Section)

In the imaging mass spectrometry method of the present invention, the sample section can be derived from any sample containing a target biological substance (e.g., a biological tissue of human, animal, or plant). The sample section may be derived from biological tissues (e.g., brain, liver, lung, kidney, prostate, ovary, spleen, lymph node, thyroid, pancreas, heart, skeletal muscle, intestine, larynx, esophagus, skin, and stomach) of a human or an animal (e.g., monkey, dog, cat, mouse, guinea pig, rat, hamster, horse, cow, pig, bird, and fish); however, these are specific examples and not limitations.

The sample section can be prepared by using a technique well known to those skilled in the art, such as a microtome. The thickness of the sample section is not limited as long as the derivatization reagent can derivatize the biological substance and can be 1 to 20 μm, for example.

### (Biological Substance)

In the imaging mass spectrometry method of the present invention, the sample section contains a biological substance. In this description, the biological substance is an endogenous substance derived from metabolism of an organism, a chemical substance derived from a microorganism symbiotic with the organism, or a drug or chemical substance ingested by or administered to the organism and metabolites thereof. The biological substance may be any substance derivatized through a reaction with the first derivatization reagent or the second derivatization reagent, and examples thereof include an endogenous substance (sugar, protein, peptide, glycoprotein, glycopeptide, nucleic acid, glycolipid, etc.), pharmaceutical compounds or candidate substances thereof, chemical substances derived from food or luxury grocery items, pesticides, and environmental pollutants. The biological substances also include substances subjected to physiological modification such as phosphorylation.

The biological substance is preferably a substance having an amino group and/or a carboxy group, more preferably an amino acid, an amino acid derivative having an amino group and/or a carboxy group, or amines. As used herein, the term “amino acid” means an organic compound having both amino group and carboxyl group as functional groups. The biological substance may be an α-amino acid, which is a constituent element of a protein or peptide, or an α-amino acid derivative having an amino group and/or a carboxy group. Strictly speaking, proline is an imino acid; however, in this description, proline is included in amino acids and α-amino acids.

In the imaging mass spectrometry method of the present invention, a physiologically active amine having an amino group, an α-amino acid, and a metabolite thereof can be used as a target biological substance. Specific examples of the physiologically active amine include γ-aminobutyric acid (GABA), L-dopa, norepinephrine, dopamine, tryptamine, serotonin, putomine, histamine, tyramine, taurine, spermidine, and spermine. Examples of α-amino acid include arginine, asparagine, aspartic acid, cysteine, glutamine, glutamic acid, glycine, histidine, isoleucine, leucine, lysine, methionine, phenylalanine, alanine, serine, threonine, tryptophan, tyrosine, valine, and proline. Examples of the metabolite of α-amino acid include kynurenine, which is a metabolite of tryptophan.

With the method of the present invention, two or more biological substances can simultaneously be derivatized and quantified. Therefore, the imaging mass spectrometry method of the present invention is not limited to one biological substance and includes quantitatively comparing the amounts of two or more biological substances between multiple tissue sections. For example, by using the mTRAQ (registered trademark) reagents as the derivatization reagents and using both GABA and dopamine as the internal standards, quantitative comparison of GABA and dopamine can be performed in multiple tissue sections in one measurement.

A biological tissue extract containing the amine/amino acid obtained by an extraction method such as the Bligh & Dyer method can be treated with the derivatization reagents to obtain an extract sample containing derivatized internal standard substances. The extract sample can be applied to tissue sections to perform a quantitative comparison of multiple tissue sections with respect to many types of biological substances in one measurement. In this case, many types of biological substances can quantitatively be compared with less labor.

### (Isotope Labeling)

As used herein, the term “isotope labeling” means that a stable isotope is used to cause a mass difference between the first derivatization reagent and the second derivatization reagent. Isotope labeling can be performed by using one or more stable isotopes such as 2H, 13C, 15N, 17O, 18O, 33P, and 34S, and 13C and/or 15N can preferably be used. Both the first derivatization reagent and the second derivatization reagent can contain isotopes of particular atoms and, for example, the first derivatization reagent can contain three 13Cs and one 15N while the second derivatization reagent can contain six 13Cs and two 15Ns. The mass of the first derivatization reagent may be less than the mass of the second derivatization reagent.

In this description, the terms “quantitatively compare”, “comparative quantitation”, and “quantitative comparison” are interchangeably used. These terms mean that the amount of biological substance is compared between multiple, for example, two, three, or four or more, tissue sections. These terms include both the comparison only between specific sites of the tissue sections and the comparison between whole tissue sections.

### (Step (A))

The imaging mass spectrometry method of the present invention includes a step of bringing a first derivatization reagent into contact with a biological substance in a first sample section. Due to the contact, the biological substance in the sample section is derivatized. A technique well known to those skilled in the art such as application or spraying can be used as a means for the contact. An amount of a first derivatization reagent or the second derivatization reagent applied or sprayed can appropriately be adjusted depending on a thickness of a sample section and a type of the biological substance. The first derivatization reagent is preferably dispersed in a solvent such as acetonitrile for uniform application or spraying. To promote derivatization, the application or spraying is preferably followed by incubation at a suitable temperature (e.g., 5 to 40° C.) for a certain period of time.

### (Step (B))

The imaging mass spectrometry method of the present invention includes a step of applying the internal standard substance derivatized with the second derivatization reagent to the first sample section. The internal standard substance generally refers to a substance added in a predetermined amount to a sample mainly to correct a variation of a quantitative value in each experiment when the substance is quantified by a mass spectrometry method etc. In the imaging mass spectrometry method of the present invention, a compound corresponding to the internal standard substance (e.g., amino acid standard) is derivatized and used. When multiple biological substances are quantified, multiple internal standard substances are used. A quantitative comparison can be made between multiple tissue sections based on a spectral intensity ratio between the biological substance and the internal standard substance. By treating the biological tissue extract with the derivatization reagent and derivatizing the substances contained in the biological tissue together, comprehensive quantitative comparison of the biological substances contained in the sample section can be performed. The biological tissue to be treated is preferably a biological tissue of an individual from which the sample section for applying the treated biological tissue is derived or may be a biological tissue of another individual.

The derivatized internal standard substance may be prepared before step (A) or may be a commercially available substance. The derivatized internal standard substance can be prepared by a method well known to those skilled in the art and can be prepared, for example, by mixing and stirring the derivatization reagent and the substance to be derivatized in triethylamine hydrogen carbonate and incubating the mixture for a period of time. As described above, to use multiple substances contained in the biological tissue extract as the internal standard substances, the substances contained in the biological tissue extract may be derivatized together.

A technique well known to those skilled in the art such as application or spraying can be used as a means for the application. In this case, an amount to be applied or sprayed can appropriately be adjusted depending on a thickness of the sample section and a type of the biological substance.

In the imaging mass spectrometry method of the present invention, the first derivatization reagent is uniformly applied onto the sample section, and the derivatized internal standard substance is also uniformly applied thereon. It is preferable that no extreme difference in quantitative ratio occurs between the first derivatization reagent and the derivatized internal standard substance.

### (Step (C))

The imaging mass spectrometry method of the present invention includes a step of bringing the biological substance and the internal standard substance into contact with a matrix to obtain a mass spectrum. The matrix means a compound used in MALDI and efficiently absorbing laser energy. In an embodiment of the imaging mass spectrometry method of the present invention, the biological substance and the internal standard substance are brought into contact with the matrix and then irradiated with a laser beam. The contact with the matrix can result in efficient ionization of the derivatized biological substance and internal standard substance during laser irradiation. To bring the biological substance and the internal standard substance into contact with the matrix, the matrix is preferably sprayed by a spray.

The matrix can appropriately be selected depending on the biological substance. Examples thereof include, but not limited to, α-cyano-4-hydroxycinnamic acid (CHCA), 2,5-dihydroxybenzoic acid (DHB), and sinapinic acid (SA). The matrix is preferably used after being dispersed in a solvent such as acetonitrile or ethanol. A trifluoroacetic acid is preferably further added to the matrix, and the concentration thereof is preferably 0.05% (v/v) or more and 0.5% (v/v) or less, further preferably 0.1% (v/v) or more and 0.3% (v/v) or less.

### (Step (D))

The imaging mass spectrometry method of the present invention includes a step of bringing the first derivatization reagent or the second derivatization reagent into contact with a biological substance in a second sample section. Although the means for the contact, the amount of the first derivatization reagent or the second derivatization reagent applied or sprayed, and the incubation temperature and time may be different from step (A), the same technique, the same amount, and the same incubation temperature and time as step (A) are preferably used. Additionally, the derivatization reagent used in step (A) is preferably used in step (D) and, for example, when the first derivatization reagent is used in step (A), the first derivatization reagent is also used in step (D).

### (Step (E))

The imaging mass spectrometry method of the present invention includes a step of applying to the second sample section the internal standard substance of the biological substance derivatized with the first derivatization reagent or the second derivatization reagent not used in (D). Although the means for the application, the amount of the first derivatization reagent or the second derivatization reagent applied or sprayed, etc. may be different from step (B), the same technique and the same amount as step (B) are preferably used.

In the imaging mass spectrometry method of the present invention, the first derivatization reagent or the second derivatization reagent is uniformly applied onto the second sample section, and the internal standard substance is also uniformly applied thereon. It is preferable that no extreme difference in quantitative ratio occurs between the derivatization reagent and the internal standard substance.

### (Step (F))

The imaging mass spectrometry method of the present invention includes a step of bringing the biological substance and the internal standard substance on the second sample section into contact with a matrix to obtain a mass spectrum. This step is preferably performed in the same way as step (C).

### (Step (G))

The imaging mass spectrometry method of the present invention includes a step of quantitatively comparing a spectral intensity ratio of the derivatized biological substance and the internal standard substance between the first sample section and the second sample section. According to the imaging mass spectrometry method of the present invention, the spectral intensity ratio of the biological substance and the internal standard substance (biological substance/internal standard substance) can be calculated by making a correction with the internal standard substance. By comparing the intensity ratio, the quantitative comparison of the biological substance between the tissue sections can accurately be performed.

The order of step (A), step (B), and step (C) or the order of step (D), step (E), and step (F) is not limited as long as the distribution information and/or quantitative information of the biological substance is obtained, and the steps can be performed in any order. However, step (A), step (B), and step (C), or step (D), step (E), and step (F), are preferably performed in this order.

Similarly, either of the operations for the first sample section (i.e., steps (A) to (C)) and the second sample section (i.e., steps (D) to (F)) may be performed first as long as the quantitative comparison of the biological substance can be performed between multiple tissue sections.

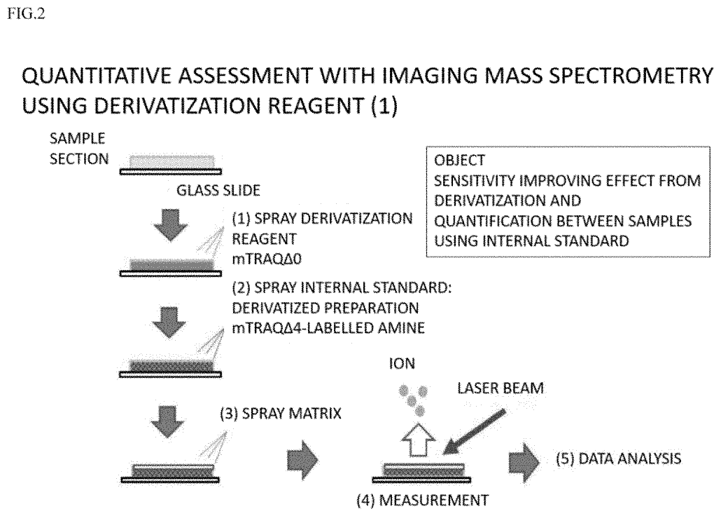

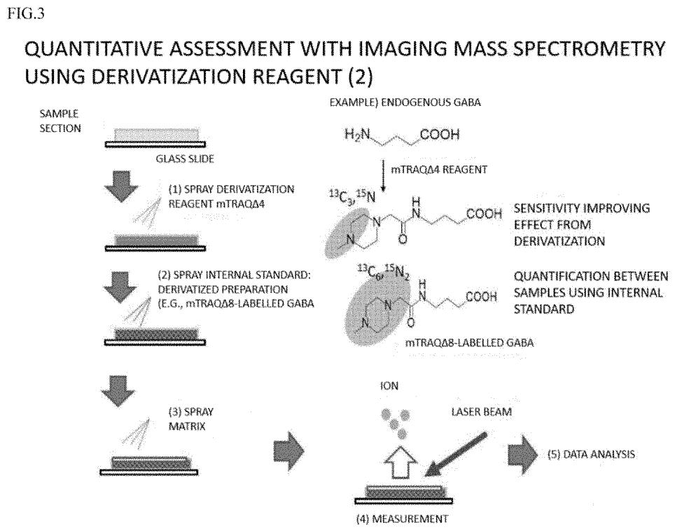

FIG. 2 shows an operation example in which an amino acid and the mTRAQ (registered trademark) reagents are used as the biological substance and the derivatization reagents, respectively, to perform the operation in the order of steps (A), (B), and (C). FIG. 3 shows an operation example in which an amino acid and the mTRAQ (registered trademark) reagents are used as the biological substance and the derivatization reagents, respectively, to perform the operation in the order of steps (A), (B), and (C). These operation examples are merely examples, and the imaging mass spectrometry method of the present invention is not construed as being limited to these operation examples.

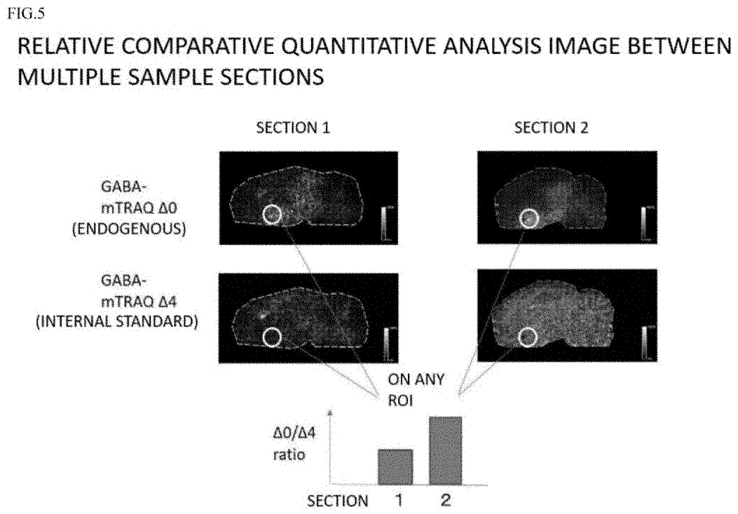

FIG. 5 shows a schematic diagram showing in the case of using GABA as the biological substance and the mTRAQ (registered trademark) reagents as the derivatization reagents to perform a quantitative comparison between two sections.

The imaging mass spectrometry method of the present invention can include (H) a step of treating a biological tissue extract with the second derivatization reagent to obtain a first extract sample containing a derivatized internal standard substance, and (I) a step of treating the biological tissue extract with the derivatization reagent used in step (E) to obtain a second extract sample containing a derivatized internal standard substance. Although the biological tissue extraction method is not particularly limited as long as the substance to be analyzed is extracted, for example, the Bligh & Dyer method can be employed.

### (Imaging Mass Spectrometry Method)

An ionization means for performing the imaging mass spectrometry method of the present invention can appropriately be selected depending on a type of the biological substance, and a matrix assisted laser desorption/ionization method (MALDI) is preferably used. Although a commercially available imaging mass spectrometer such as MALDI-FTMS manufactured by Bruker can be used for the analysis with the imaging mass spectrometry, the present invention is not limited thereto.

### [2] Quantification Kit

A kit of the present invention includes the first derivatization reagent for derivatizing the biological substance, and the internal standard substance of the biological substance derivatized with the second derivatization reagent. The kit can also include instructions for use. The kit may include optional constituent elements such as a buffer, a stabilizer, and a reaction container.

### EXAMPLES

### Example 1: Brain GABA Measurement Test of Stroke Model Rat Using Imaging Mass Spectrometry

1. Collection and Sectioning of Brain Samples Derived from Stroke Model Rat and Control Rat

1) A stroke model rat (Japan SLC, Inc.) and a control rat (Japan SLC, Inc.) were decapitated at 9 weeks of age (without anesthesia) and the whole brain was promptly removed to minimize postmortem degradation of biological substances in the brain.

2) The removed whole brain was washed with saline, drained, wrapped in aluminum foil, and frozen with liquid nitrogen. The frozen brain was then stored at −80° C.

3) By using Cryomicrotome CM3050S (manufactured by Leica), brain sections of the sagittal plane were prepared at 10 μm, and two brain sections were affixed to one slide glass. Ten slide glasses were prepared for each individual.

4) The slide glass having the sections attached thereto was put in a 50 mL tube containing silica gel and stored at −80° C. Only one hemisphere of the brain was sliced, and the remaining hemisphere was continuously stored at −80° C.

2. Preparation of mTRAQΔ0 Solution for Endogenous GABA Derivatization

1) To 1 vial of the mTRAQΔ0 reagent (manufactured by SCIEX), 40 μL of acetonitrile (manufactured by Kanto Kagaku) was added and stirred.

2) To 20 μL of the solution prepared at 1), 20 μL of TEAB (manufactured by Sigma-Aldrich) was added and stirred.

3. Preparation of mTRAQΔ4 Solution for Internal Standard GABA Derivatization

1) GABA standard (manufactured by Sigma-Aldrich) was dissolved in test water to prepare a 1 mg/mL GABA standard solution.

2) To 30 μL of the solution prepared at 1), 30 μL of triethylamine hydrogen carbonate (manufactured by Sigma-Aldrich) was added, and 20 μL of mTRAQΔ4 reagent (manufactured by SCIEX) was further added and stirred.

3) Incubation (1 hour, room temperature) was performed.

4) Twenty microliters of 1.2% hydroxylamine (manufactured by Tokyo Chemical Industry Co., Ltd.) was added.

### 4. Preparation of Matrix Solution

1) Trifluoroacetic acid (Kanto Chemical Co., Inc.) was added to 70% ethanol (Kanto Chemical Co., Inc.) to prepare 70% ethanol containing 0.2% TFA.

2) DHB (manufactured by Sigma-Aldrich) was dissolved in 0.1% TFA-containing ethanol to prepare a 30 mg/mL DHB solution, which was used as a matrix solution.

### 5. Treatment of Sample Sections

1) The mTRAQΔ0 solution for endogenous GABA derivatization was applied to the brain section by using a spray device (ImagePrep, manufactured by Bruker Daltonics).

2) Incubation (1 hour, 33° C., under water vapor pressure for test) was performed.

3) The derivatized internal standard GABA solution prepared at 3. was applied to the brain section by using the spray device.

4) The matrix solution was applied to the brain section by using the spray device.

5) The sample obtained at 4) was used as an imaging mass spectrometry measurement sample.

### 6. Measurement

Measurement was performed by using solariX XR (manufactured by Bruker Daltonics) in full scan (m/z: 100-500) at a spatial resolution of 200 μm.

### 7. Data Analysis

Distribution and quantitative analyses of mTRAQΔ0-GABA (m/z: 244.1656) and mTRAQΔ4-GABA (m/z: 248.1726) were performed by using flexImaging MS Software, SCiLS Lab, Microsoft Excel, EXSUS, and Prism.

### 8. Measurement Result 1 (Quantitative Comparison Between Two Sample Sections)

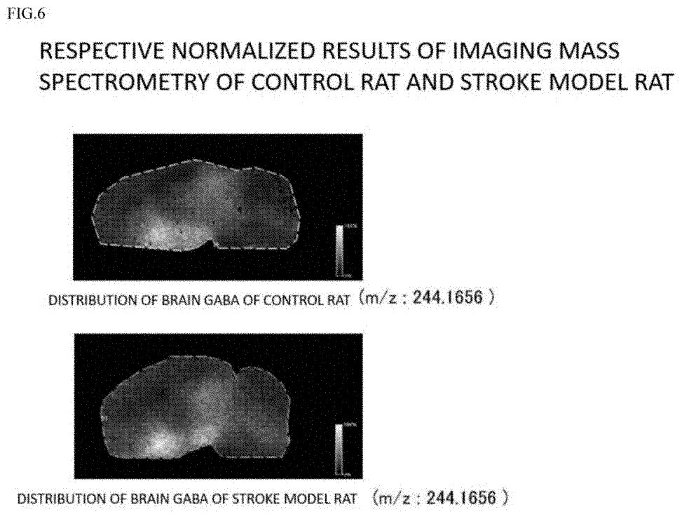

Table 1 below shows peak areas around the amygdalas of the control and stroke model rats. FIG. 6 shows respective results of the imaging mass spectrometry of the control rat and the stroke model rat. On the sections shown in images, a whiter portion shows a higher concentration of the biological substance, and a darker portion shows a lower concentration of the biological substance.

TABLE 1
Peak Areas around Amygdalas of Control and Stroke Model Rats

| | mTRAQΔ0 | mTRAQΔ4 | mTRAQΔ0/Δ4 |
|---|---|---|---|
| control | 14998 | 19920 | 0.75 |
| Stroke | 1551 | 3702 | 0.42 |

It is known that GABA accumulates around the amygdala in rats. As compared to the control rat, the GABA concentration in the stroke model rat tended to be reduced, and the concentration was about 0.56 times. From the above, it was demonstrated that a quantitative comparison of GABA can be performed between the control rat sample section and the stroke model rat sample section.

### Example 2: Comprehensive Analysis of Multiple Substances by Batch Derivatization of Amino Group-Containing Hydrophilic Metabolites in Brain Homogenate Extract

In Example 2, amino group-containing hydrophilic metabolites in a brain homogenate extract were used as the internal standard substances. Specifically, the amino group-containing hydrophilic metabolites in the brain homogenate extract were collectively derivatized with mTRAQΔ4 and used as the internal standard substances to comprehensively analyze 16 types of biological substances in the sample section.

1. Collection and Sectioning of Brain Samples Derived from Stroke Model Rat

The same procedure as 1. of Example 1 was used for collection and sectioning of brain samples derived from a stroke model rat.

2. Preparation of mTRAQΔ0 Solution for Derivatization of Amino Group-Containing Hydrophilic Metabolites (Biological Substances) in Sample Section

The same procedure as 2. of Example 1 was used.

3. Preparation of Rat Brain Homogenate Extract and Preparation of mTRAQΔ4 Solution for Derivatization of Amino Group-Containing Hydrophilic Metabolites (Internal Standard Substances) Contained in Brain Homogenate Extract

1) The dried rat brain homogenate extract was dissolved in test water to prepare a rat brain homogenate solution.

2) To 30 μL of the solution prepared at 1), 30 μL of triethylamine hydrogen carbonate (manufactured by Sigma-Aldrich) was added, and 20 μL of mTRAQΔ4 reagent (manufactured by SCIEX) was further added and stirred.

3) Incubation (1 hour, room temperature) was performed.

4) Twenty microliters of 1.2% hydroxylamine (manufactured by Tokyo Chemical Industry Co., Ltd.) was added.

### 4. Preparation of Matrix Solution

The same procedure as 4. of Example 1 was used.

### 5. Treatment of Sample Sections

1) The mTRAQΔ0 solution for biological substance derivatization was applied to the brain section by using a spray device (ImagePrep, manufactured by Bruker Daltonics).

2) Incubation (1 hour, 33° C., under water vapor pressure for test) was performed.

3) The sample prepared at 3. was applied by using the spray device.

4) The matrix solution was applied to the brain section by using the spray device.

5) The sample obtained at 4) was used as an imaging mass spectrometry measurement sample.

### 6. Measurement

Measurement was performed by using the same procedure as 6. of Example 1.

### 7. Data Analysis

The amino group-containing hydrophilic metabolites derivatized with mTRAQΔ0 (derivatized biological substances) and the amino group-containing hydrophilic metabolites derivatized with mTRAQΔ4 (derivatized internal standard substances) were analyzed by using flexImaging MS Software, SCiLS Lab, Microsoft Excel, EXSUS, and Prism. The m/z values of each of the amino group-containing hydrophilic metabolites is as follows.

TABLE 2

| | mTRAQΔ0 | mTRAQΔ4 |
|---|---|---|
| glycine | 216.1343 | 220.1414 |
| alanine | 230.1499 | 234.1570 |
| GABA | 244.1656 | 248.1727 |
| serine | 246.1448 | 250.1519 |
| glutamine | 287.1714 | 291.1785 |
| glutamic acid | 288.1554 | 292.1625 |
| threonine | 260.1605 | 264.1676 |
| leucine | 272.1969 | 276.2040 |
| aspartic acid | 274.1397 | 278.1468 |
| arginine | 315.2139 | 319.2210 |
| proline | 256.1656 | 260.1727 |
| phenylalanine | 306.1812 | 310.1883 |
| lysine | 287.2078 | 291.2149 |
| methionine | 290.1533 | 294.1604 |
| tyrosine | 322.1761 | 326.1832 |
| histidine | 296.1717 | 300.1788 |

### 8. Measurement Result 2

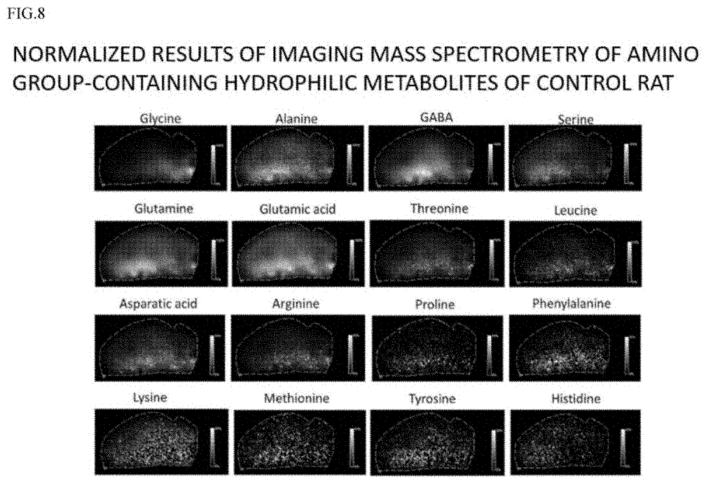

FIG. 8 shows a result of the imaging mass spectrometry of the control rat. FIG. 8 shows images of distribution normalized based on the spectral intensity ratio between the derivatized biological substance and the derivatized internal standard substance. On the sections shown in the images, a whiter portion shows a higher concentration of the biological substance, and a darker portion shows a lower concentration of the biological substance. As shown in FIG. 8, the images of distribution normalized based on the spectral intensity ratio between the derivatized biological substance and the derivatized internal standard substance were obtained for all the 16 types of the amino group-containing hydrophilic metabolites in the control rat. By using the obtained images as controls, comparative quantification can be performed for a pathological rat.

### INDUSTRIAL APPLICABILITY

The present invention provides the imaging mass spectrometry capable of not only providing distribution information of a biological substance in a tissue but also enabling quantitative comparison of amounts of biological substances between multiple tissue sections. According to the present invention, the amounts of the biological substances can quantitatively be compared between a tissue section of a pathological model mouse and a tissue section of a normal model mouse.

1. An imaging mass spectrometry method of a biological substance multiple sample sections, the method comprising the steps of:
(A) bringing a first derivatization reagent into contact with the biological substance in a first sample section;
(B) applying an internal standard substance of the biological substance to the first sample section, the internal standard substance being derivatized with a second derivatization reagent;
(C) bringing the biological substance and the internal standard substance on the first sample section into contact with a matrix to obtain a mass spectrum;
(D) bringing the first derivatization reagent or the second derivatization reagent into contact with the biological substance in a second sample section;
(E) applying the internal standard substance of the biological substance to the second sample section, the internal standard substance being derivatized with the first derivatization reagent or the second derivatization reagent not used in the step (D);
(F) bringing the biological substance and the internal standard substance on the second sample section into contact with the matrix to obtain the mass spectrum; and
(G) quantitatively comparing a spectral intensity ratio of the derivatized biological substance and the derivatized internal standard substance between the first sample section and the second sample section,
wherein the second derivatization reagent is the first derivatization reagent labeled with an isotope.

2. The imaging mass spectrometry method according to claim 1, wherein the first derivatization reagent is used in the step (D), and wherein the second derivatization reagent is used in the step (E).

3. The imaging mass spectrometry method according to claim 1, wherein the first derivatization reagent and the second derivatization reagent are an amino group modification reagent respectively, and wherein the biological substance in the first and second sample sections is a substance having an amino group.

4. The imaging mass spectrometry method according to claim 3, wherein the substance having the amino group is an α-amino acid or a derivative thereof.

5. The imaging mass spectrometry method according to claim 3, wherein the first derivatization reagent and the second derivatization reagent are mTRAQ (registered trademark) reagents having different molecular weights respectively.

6. The imaging mass spectrometry method according to claim 1, wherein the sample sections are derived from human or animal biological tissues.

7. The imaging mass spectrometry method according to claim 6, further comprising:
(H) a step of treating a biological tissue extract with the second derivatization reagent to obtain a first extract sample containing a derivatized internal standard substance, and
(I) a step of treating the biological tissue extract with the derivatization reagent used in the step (E) to obtain a second extract sample containing the derivatized internal standard substance,
wherein the first extract sample is applied to the first sample section in the step (B), and wherein the second extract sample is applied to the second sample section in the step (E).

8. The imaging mass spectrometry method according to claim 1, wherein two or more internal standard substances are applied to the first sample section in the step (B), and wherein two or more internal standard substances are applied to the second sample section in the step (E).

9. A quantification kit for a biological substance in a living body, comprising:
a first derivatization reagent for labeling and derivatization of the biological substance; and
an internal standard substance of the biological substance, which is derivatized with a second derivatization reagent,
wherein an imaging mass spectrometry is used for quantification of the biological substance, and wherein the second derivatization reagent is the first derivatization reagent labeled with an isotope.

10. The quantification kit according to claim 9, wherein the first derivatization reagent and the second derivatization reagent are an amino group modification reagent respectively, and wherein the biological substance in the living body is a substance having the amino group.

11. The quantification kit according to claim 10, wherein the substance having the amino group is an amino acid or a derivative thereof.

12. The quantification kit according to claim 10, wherein the first derivatization reagent and the second derivatization reagent are mTRAQ (registered trademark) reagents having different molecular weights respectively.

13. The imaging mass spectrometry method according to claim 2, wherein the first derivatization reagent and the second derivatization reagent are an amino group modification reagent respectively, and wherein the biological substance in the first and second sample sections is a substance having an amino group.

14. The imaging mass spectrometry method according to claim 4, wherein the first derivatization reagent and the second derivatization reagent are mTRAQ (registered trademark) reagents having different molecular weights respectively.

15. The imaging mass spectrometry method according to claim 2, wherein the sample sections are derived from human or animal biological tissues.

16. The imaging mass spectrometry method according to claim 3, wherein the sample sections are derived from human or animal biological tissues.

17. The imaging mass spectrometry method according to claim 4, wherein the sample sections are derived from human or animal biological tissues.

18. The imaging mass spectrometry method according to claim 5, wherein the sample sections are derived from human or animal biological tissues.

19. The imaging mass spectrometry method according to a claim 2, wherein two or more internal standard substances are applied to the first sample section in the step (B), and wherein two or more internal standard substances are applied to the second sample section in the step (E).

20. The imaging mass spectrometry method according to a claim 3, wherein two or more internal standard substances are applied to the first sample section in the step (B), and wherein two or more internal standard substances are applied to the second sample section in the step (E).
